(19) United States
(12) Patent Application Publication
(10) Pub. No.: US 2011/0052497 A1
(43) Pub. Date: Mar. 3, 2011

(54) Minimally Invasive Systems and Methods for In Vivo Testing of Materials

(75) Inventor: Heejin Lee, Arlington, MA (US)

(73) Assignee: TARIS BIOMEDICAL, INC., Lexington, MA (US)

(21) Appl. No.: 12/870,261

(22) Filed: Aug. 27, 2010

Related U.S. Application Data
(60) Provisional application No. 61/237,517, filed on Aug. 27, 2009.

(51) Int. Cl. A61K 49/00; C12Q 1/02; C12M 1/34; G01N 31/00

(52) U.S. Cl. 424/9.1; 435/29; 435/287.1; 436/5

(57) ABSTRACT

Implantable material testing devices and method of testing materials in an animals are provided. In one embodiment, a method of testing a material in an animal includes associating the material with a retention frame to form a testing device. The retention frame movable between a first shape suited for insertion through a deployment instrument and a second shape suited for retention in the animal. The testing device is inserted in the first shape into the deployment instrument and is driven through the deployment instrument into the animal. Once in the animal, the testing device is permitted to assume the second shape so that the testing device is retained in the animal for a testing period. The testing device is removed from the animal after the testing period is complete, and the material is analyzed. The material may be analyzed for biofilm formation, encrustation, or degradation. The animal also may be analyzed for infection or other reactions to the material. Tissue may be collected from the animal, and the tissue may be analyzed. The testing period may be between about 1 day and about 90 days.

### CROSS-REFERENCE TO RELATED APPLICATIONS

This application claims the benefit of U.S. Provisional Application No. 61/237,517, filed Aug. 27, 2009, which is incorporated herein by reference in its entirety.

### BACKGROUND OF THE INVENTION

Genitourinary devices and equipment are typically formed from materials that are suited for use in the genitourinary system. For example, urological devices and equipment such as ureteral stents, urethral stents, and urethral catheters often include materials such as natural or synthetic polymeric compounds, coatings, and surface treatments. To investigate the suitability of these materials for use in humans, these materials are often tested before human use to ensure biocompatibility and to identify the risk of undesirable effects such as biofilm formation, infection, and encrustation. Typically, both in vitro analysis and in vivo animal testing are performed. In vivo animal testing usually involves implanting the materials in animal models, such as rabbit and pig. The materials are often surgically implanted in the animal bladder, such as via open cystotomy. However, surgically implanting the materials is cumbersome and causes trauma to the animal. The animal also may have difficulty retaining the material in the bladder without accidental voiding, which disrupts the study.

Thus, a need exists for systems and methods of in vivo testing of materials in animals, such as in the bladder or other body cavity of a mammalian laboratory animal. A need also exists for systems and methods that permit deploying the test material into the animal through a natural lumen of the animal in a minimally invasive procedure. In addition, the systems and methods should be able to retain the test material in the animal without the animal voiding the material from the bladder or other body cavity.

### SUMMARY

Implantable material testing devices and method of testing materials in an animals are provided. In one embodiment, a method of testing a material in an animal includes associating the material with a retention frame to form a testing device. The retention frame can be moved between a first shape and a second shape, the first shape for insertion through a deployment instrument and the second shape for retention in the animal. The testing device is inserted into the deployment instrument in the first shape and is driven through the deployment instrument into the animal. Once in the animal, the testing device is permitted to assume the second shape so that the testing device is retained in the animal for a testing period. The testing device is removed from the animal after the testing period is complete, and the material is analyzed. For example, the material may be analyzed for biofilm formation, encrustation, or degradation. The animal also may be analyzed for infection or other reactions to the material. Tissue may be collected from the animal, and the tissue may be analyzed. The testing period may be between about 1 day and about 90 days.

In another embodiment, a method for minimally invasive in vivo testing of a material includes deploying a testing device into the bladder of a mammalian laboratory animal, the testing device including one or more test materials. The testing device is deployed via a deployment instrument inserted through the urethra. The device is left in the bladder for a period effective to achieve a study objective concerning the test material or materials, such as a period from 1 day to 90 days. The testing device is then removed from the bladder, such as via a deployment instrument inserted through the urethra or via necropsy. The test material or materials may be analyzed for biofilm formation, encrustation, or degradation, and the animal may be analyzed for infection or other reactions to the test material or materials. For example, tissue may be collected from the bladder and analyzed.

In another embodiment, a material testing device is provided for testing a material in a body cavity of an animal. The material testing device includes a retention frame portion and at least one material portion. The retention frame portion is suited for retaining the device in the body cavity. The material portion is associated with the retention frame portion for testing in the body cavity.

The material portion may include a first material portion and a second material portion, the first material portion differing from the second material portion in size, shape, surface texture, surface treatment, porosity, or some combination thereof. The first and second material portions may be discrete units. At least one material portion may include a polymeric material.

The retention frame portion may include an elastic wire. The retention frame portion also may include a superelastic alloy or shape memory material. For example, the retention frame portion may include a nitinol wire. The retention frame portion may have a spring constant in the range of about 3 N/m to about 4 N/m. The retention frame portion also may include one or more windings, coils, spirals, loops, curls or sub-circles.

### BRIEF DESCRIPTION OF THE DRAWINGS

FIG. 1 is a side view of an embodiment of a material testing device.

FIG. 2 is a side view of the embodiment of the device shown in FIG. 1, illustrating the device in a deployment instrument for implantation in an animal.

FIG. 3 is a side view of an example material testing device, illustrating an example size of the material testing device in comparison to an approximation of the bladder trigone region.

FIG. 4 illustrates examples of shapes for a retention frame portion of a material testing device.

FIG. 5 illustrates examples of shapes for a material portion of a material testing device.

FIG. 6 is a side view of an embodiment of a material testing device associated with a number of material portions of various configurations.

FIG. 7 illustrates various material testing devices to illustrate various associations of a material portion and a retention frame portion.

FIG. 8 is a block diagram illustrating an embodiment of a method of making a material testing device.

FIG. 9 is a sagittal view of a human male, illustrating a material testing device exiting a deployment instrument into a bladder of the male.

FIG. 10 illustrates a material testing device assuming a retention shape as it exits a deployment instrument.

### DESCRIPTION OF THE INVENTION

Described below are systems and methods of in vivo testing of materials. In embodiments, a material testing device includes a material portion and a retention frame portion. The material portion includes one or more materials to be tested in vivo. The retention frame portion is associated with the material portion to retain the material portion in the body.

The material testing device may be deployed into a genitourinary site of a body for the purpose of testing one or more materials in the body. The term “genitourinary site” denotes a site within a urological or reproductive system of the body, including a bladder, kidney, urethra, ureter, penis, testicle, seminal vesicle, vas deferens, ejaculatory duct, prostate, vagina, uterus, ovary, or fallopian tube, among others or combinations thereof. In some embodiments, the material testing device is deployed in a urological system to test a urological material. In particular embodiments, the material testing device is deployed in the bladder. However, the material testing device can be deployed in other body cavities, tissues, or lumens in other embodiments, including those outside of the genitourinary systems.

The material testing device may be deployed into the body of any subject to test the material in vivo. The subject may be a human or animal subject, whether adult or child, male or female. In particular embodiments, the subject is an animal, such as a rabbit, pig, cat, dog or sheep.

The material may include a biomaterial that is being tested for use in the body, such as a urological material or other material intended for use in a genitourinary site. The material to be tested may be one intended for use as a material of construction or coating material for a medical device designed for short term or long term deployment in vivo, such as in the bladder. The material may be a polymer, a metal, a ceramic, or a composition of one or more of these. Other materials also may be tested. Characteristics of a material also may be tested, such as the effect of its size, shape, surface texture, surface treatment, or porosity, among others or combinations thereof, on its suitability for use in the body. The relationship between two or more materials or surface treatments also may be tested.

The material testing device may be deployed into the subject in a minimally invasive implantation procedure. The term “minimally invasive” indicates the implantation procedure is relatively less invasive than conventional (e.g. open) surgical procedures. In some embodiments, the device is at least partially implanted through a natural lumen of the body, such as through the urethra. In particular embodiments, the device is implanted without surgical incision. In preferred embodiments, for example, the device is deployed through the urethra into the bladder.

The material testing device may be used to test materials locally in essentially any site of an animal. In one embodiment, a material testing method includes forming a material testing device; implanting the material testing device in the animal; retaining the material testing device in the animal for the duration of the test period using the retaining portion; removing the material testing device from the animal after the testing period is complete; and analyzing the material. Examples of characteristics that may be tested include biofilm formation, encrustation, degradation, bacterial adhesion, colonization, biocompatibility, biodegradability, mechanical integrity, strength, and resistance to erosion, among others. The animal also may be analyzed, such as for infection, tolerance to the material, thrombogenicity, or other reaction to the material. These tests of characteristics may be performed by visual observation with or without the aid of standard laboratory equipment.

The devices and methods disclosed herein build upon those described in the following U.S. patent applications, which are incorporated by reference herein: U.S. application Ser. No. 11/463,956, filed Aug. 11, 2006; U.S. application Ser. No. 12/333,182, filed Dec. 11, 2008; U.S. application Ser. No. 12/538,580, filed Aug. 10, 2009; U.S. application Ser. No. 12/825,215, filed Jun. 28, 2010; U.S. application Ser. No. 12/825,238, filed Jun. 28, 2010; U.S. application Ser. No. 12/851,494, filed Aug. 5, 2010; U.S. Provisional Application No. 61/241,229, filed Sep. 10, 2009; U.S. Provisional Application No. 61/287,649, filed Dec. 17, 2009; and U.S. Provisional Application No. 61/311,103, filed Mar. 5, 2010.

### I. The Device

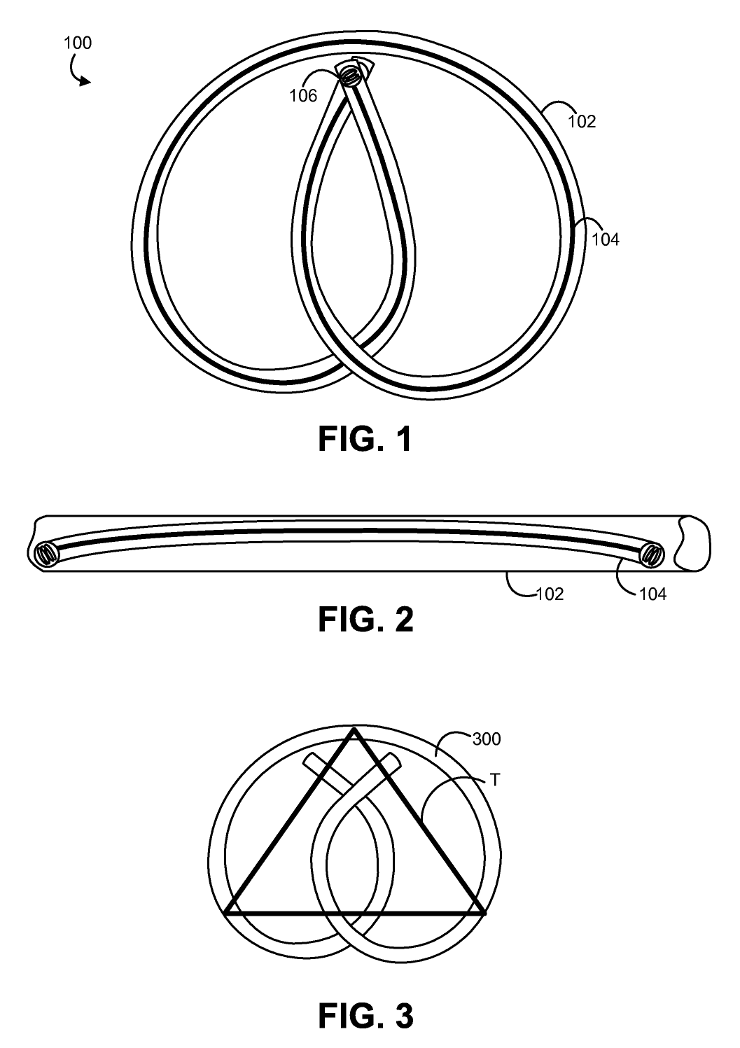

FIG. 1 illustrates an example embodiment of a material testing device 100, the device having both a material portion 102 and a retention frame portion 104. The material portion 102 includes a material to be tested in the body. For example, the material portion 102 may include a urological material that is being investigated for use in a urological application, such as in a urethral stent, a ureteral stent, or a urethral catheter. The retention frame portion 104 retains the material portion 102 in the implantation site, such as a portion of a genitourinary system, during the duration of the testing. For example, the retention frame portion 104 may be suited for retaining the device 100 in a bladder of an animal, such as a rabbit or a pig, for the duration of a study. In embodiments in which the device 100 is designed for implantation in the bladder, the retention frame portion 104 impedes voiding of the device 100, and thus the material portion 102, from the bladder.

The retention frame portion 104 may be flexible so that the device can be deformed for insertion, yet the retention frame portion 104 may cause the device 100 to resist excretion once implanted. More specifically, the retention frame portion 104 may permit elastically deforming the device 100 between a deployment shape and a retention shape. The term “deployment shape” generally denotes any shape suited for deploying the material testing device 100 into the body, while the term “retention shape” generally denotes any shape suited for retaining the device in the intended implantation location, such as the bladder. For example, the deployment shape may be a relatively lower profile shape suited for deploying the device 100 through a working channel of a catheter, cystoscope, or other deployment instrument positioned in a natural lumen of the body, such as the urethra. An example relatively lower profile shape is the linear or elongated shape shown in FIG. 2, which illustrates the device 100 of FIG. 1 in a working channel 200 of a deployment instrument. The retention shape may be a relatively higher profile or expanded shape suited for retaining the device in the body. An example retention shape is the pretzel shape shown in FIG. 1, which may be suited for retaining the device in the bladder despite the contraction of the detrusor muscle or the hydrodynamic forces associated with urination.

In some embodiments, the device may naturally assume the retention shape and may be elastically deformed into the deployment shape for insertion into the body. Following passage into the body, the device may spontaneously or naturally return to the initial retention shape for retention within the body.

The device may be designed to be inserted and retrieved through the urethra, such as in embodiments in which the device is designed to be implanted in the bladder. The device may be sized and shaped to fit through the working channel of a deployment instrument positioned in the urethra, such as a narrow tubular lumen within a catheter or cystoscope. An available size or inner diameter of the deployment instrument may vary depending on the species in which the device is implanted. Typically, a cystoscope for an adult human has an outer diameter of about 5 to 7 mm, and a working channel of the cystoscope has a diameter of about 2 to 3 mm. Common urethral catheter or cystoscope have outer diameters that range in size from about 8 to 12 Fr (about 2.7 to 4 mm) for rabbit; about 3.5 to 5 Fr (about 1.17 to 1.67 mm) for cat; about 6 to 12 Fr (about 2 to 4 mm) for dog; about 8 to 10 Fr (about 2.7 to 3.3 mm) for pig; and about 8 to 14 Fr (about 2.7 to 4.7 mm) for sheep. Thus, the device may be relatively small in size. The device also may vary in size depending on the animal into which the material is to be implanted.

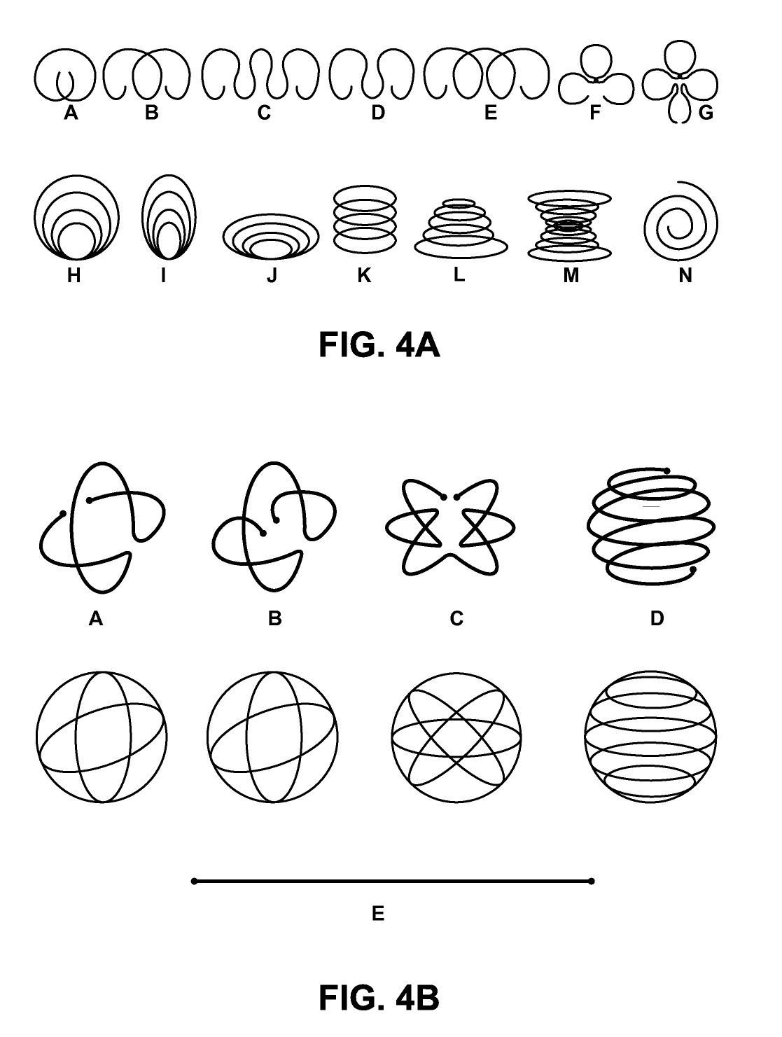

In addition to facilitating insertion, the relatively small size of the device also may reduce discomfort and trauma to the implantation site. In embodiments in which the device is implanted in the bladder, for example, the relatively small size of the device may reduce irritation of the bladder trigone, which is responsible for creating the sensation of urgency of urination. However, the overall size of the device may be larger than the bladder trigone area so that the device cannot become confined or trapped within the trigone area. For example, a bladder of an adult human typically has a capacity of about 500 mL and may have a diameter of about 12.6 cm when full. The trigone region can be approximated as a triangle having a top vertex that represents the bladder neck and two bottom vertices that represent the ureteral orifices. FIG. 3 shows an example triangle T that approximates the trigone of an adult human male. In a male, the distance from the bladder neck to one of the ureteral orifices is about 2.75 cm and the distance between the two ureteral orifices is about 3.27 cm. Thus, in FIG. 3, the distance from the top vertex to either of the bottom vertices is about 2.8 cm, while the distance between two bottom vertexes is about 3.3 cm. (The figure is merely a schematic representation of these sizes.) The device is sized so that when the device 300 overlays the triangle T, substantially the entire triangle T fits within an interior of the device 300. Such sizing ensures the device cannot become trapped in the trigone region. Of course, the size of the device can be varied depending on the size of the animal and the corresponding trigone region. In an adult female, for example, the distance between the two ureteral orifices is about 2.68 cm and the distance from a neck of the bladder to one of the ureteral orifices is about 2.27 cm. Smaller animals may have smaller trigone regions. The device also may have other sizes with respect to the trigone region.

The device also may have a density that is less than the density of urine or water, so that the device may float inside the bladder. Such floatation, although not required, may prevent the device from touching the sensitive trigone region of the bladder near the bladder neck. For example, the device may be formed from relatively low density materials of construction, or air or other gas may be entrapped in the device. The outer surface of the device preferably is soft and smooth without sharp edges or tips.

The exact configuration and shape of the device may be selected depending upon a variety of factors including the specific site of implantation, the route of implantation, the material to be tested, and the duration of the test, among others. Preferably, the design of the device will minimize pain and discomfort, while permitting retention of the material in the animal for the duration of the study. The device may be at least partially non-resorbable, such that the device may be removed at the conclusion of the study to evaluate the material samples.

The Retention Frame Portion

As mentioned above, the device includes a retention frame portion. The retention frame portion is associated with the material portion and permits retaining the material portion in the body, such as in the bladder. The retention frame portion may be elastically deformable between the retention shape and the deployment shape. For example, the retention frame may naturally assume the retention shape, may be manipulated into the deployment shape for insertion into the body, and may spontaneously return to the retention shape upon release from the deployment instrument. Thus, the device may be deployed into the body through a deployment instrument placed in a natural lumen, such as the urethra, and upon implantation the device may be retained in the body even when exposed to expected forces. For example, the device may remain in the bladder when exposed to the hydrodynamic forces associated with urination or contraction of the detrusor muscle. An example of such an embodiment is shown in FIGS. 1-2, wherein the retention frame portion assumes a pretzel shape when in the expanded position, and the retention frame portion assumes a relatively elongated, linear shape when in the lower profile position.

To achieve such a result, the retention frame portion may have an elastic limit, modulus, and/or spring constant selected to impede the device from assuming the deployment shape once implanted, limiting or preventing accidental expulsion while allowing the device to be introduced into the body in a deployment shape. Due to the properties of the retention frame portion, the device may function as a spring. Thus, the device may deform in response to a compressive load but may spontaneously return to its initial shape once the load is removed.

In a preferred embodiment, the retention frame includes an elastic wire. In one embodiment, the elastic wire comprises a superelastic alloy or other shape memory material, which are known in the art. For example, the superelastic alloy may comprise a biocompatible nickel-titanium alloy (e.g., Nitinol) or a titanium-molybdenum alloy (e.g., Flexium). In embodiments in which the retention frame comprises a shape memory material, the material used to form the frame may “memorize” the retention shape and may spontaneously assume the retention shape, upon the application of heat to the device, such as when exposed to body temperatures, or upon exiting the deployment instrument. Biodegradable, biocompatible shape memory polymers are described in U.S. Pat. No. 6,160,084 to Langer et al.

In another embodiment, the elastic wire is or includes a relatively low modulus or low durometer elastomer. Low modulus or low durometer elastomers may be relatively less likely to cause irritation to the bladder or to cause an ulcer once implanted. Some low modulus elastomers may be biodegradable. A biodegradable polymer that maintains its mechanical integrity without significant degradation in vivo can be used for testing purposes. Examples of low modulus elastomers include polyurethane, silicone, styrenic thermoplastic elastomer, and poly(glycerol-sebacate) (PGS). The elastic wire may be coated with a biocompatible polymer, such as a coating formed from one or more of silicone, polyurethane, styrenic thermoplastic elastomer, Silitek, Tecoflex, C-flex, and Percuflex, either completely or along an exposed portion that is not covered with the test material. The elastic wire may function as a spring, deforming in response to a compressive load but spontaneously returning to its initial shape once the load is removed.

The retention frame portion also may include a radio-opaque material, which may improve the visibility of the device to x-ray or other imaging techniques. The radio-opaque material may be a platinum wire wound about a portion of the device, although other radio-opaque materials can be used. The opposing ends of the retention frame portion may be adapted to avoid tissue irritation and scarring. For example, the ends may be soft, blunt, inwardly directed, joined together, or a combination thereof.

An example embodiment of a retention frame portion 104 is shown in FIGS. 1-2. The retention frame portion 104 includes an elastic wire formed from a superelastic alloy. The elastic wire may be, for example, a nitinol wire. The material portion 102 may extend along and substantially surround at least a portion of the elastic wire. The retention frame portion 104 may cause the device to assume a retention shape shown in FIG. 1, wherein the device 100 occupies an area having dimensions suited to impede expulsion of the device from the bladder of the selected animal model. The retention frame portion 104 also may permit the device to assume the deployment shape, shown in FIG. 2, wherein the device 100 may occupy an area suited for insertion through a deployment instrument 200. A radio-opaque material 108, such as a platinum wire, may be positioned about a portion of the retention frame portion, such as wound about its end. A smoothening material, such as a ultraviolet-curable epoxy, may be applied to the ends of the retention frame portion 104 to retain the radio-opaque material 108 in place and to reduce the bluntness of the retention frame portion ends. End portions of the wire may be relatively straight to facilitate passage through the biomaterial to be tested.

In embodiments in which the retention frame portion assumes a pretzel shape, the retention frame may be relatively resistant to compressive forces. A pretzel shape essentially comprises two sub-circles, each sub-circle having its own smaller arch and the sub-circles sharing a common larger arch. When the two sub-circles are first compressed together, the largest arch absorbs the majority of the compressive force and begins deforming. With continued application of the compressive force, the smaller arches of the two sub-circles overlap. Subsequently, all three of the arches resist the compressive force. The resistance to compression of the device as a whole increases once the two sub-circles overlap. Such a configuration may prevent collapse of the device, such as when the bladder contracts during urination, impeding voiding from the bladder. Additional information is provided in U.S. patent application Ser. No. 12/333,182, which is incorporated by reference herein as mentioned above.

The retention frame portion also may have shapes other than the pretzel shape shown in FIG. 1. FIGS. 4A and 4B illustrates other examples of shapes for the retention frame portion. Each of the illustrated retention frame portions has one or more windings, coils, or spirals. The retention frame may have a two-dimensional structure that is confined to a plane, or the retention frame portion may have a three-dimensional structure, such as a structure that occupies the interior of a spheroid.

Some examples are shown in FIG. 4A. In some embodiments, the frame may be in a curled configuration, such as in a configuration comprising one or more loops, curls or sub-circles. The curls may be integrally connected in a linear fashion, as shown in Examples B, C, D, and E, or in a radial fashion, as shown in Examples F and G. The curls may turn in the same direction, as shown in Examples B and E, or in alternating directions as shown in Examples C and D. The curls may also overlap, as shown in Examples A, B, and E. The frame may also include a one or more circles or ovals arranged in a two-dimensional or a three-dimensional configuration. The frame may include a number of circles, as shown in Example H, or a number of ovals, as shown in Examples I and J. Each of the circles or ovals may be closed, and the circles or ovals may be joined at a common connecting point. Alternatively, one or more of the circles and ovals may be open. The circles and ovals may also be connected at a number of connecting points. The frame also may include a number of overlapping circles or ovals. The overlapping circles or ovals each may be substantially the same size, as shown in Example K, or the circles or ovals may vary in size, as shown in Examples L and M. Circles also may be combined with ovals, depending on the embodiment. Further, the frame may be a two- or three-dimensional spiral, winding, coil, or spring having open or closed ends, as shown in Examples L, M, and N, for example.

The retention frame portion also may be a three-dimensional structure that is shaped to occupy or wind about a spheroid-shaped space, such as a spherical space, a space having a prorate spheroid shape, or a space having an oblate spheroid shape. Examples of retention frame portions that are shaped to occupy or wind about a spherical space are shown in FIG. 4B, with each retention frame portion shown above a representation of the frame in a sphere. The retention frame portion may generally take the shape of two intersecting circles lying in different planes as shown in Example A, two intersecting circles lying in different planes with inwardly curled ends as shown in Example B, three intersecting circles lying in different planes as shown in Example C, or a spherical spiral as shown in Example D. In each of these examples, the retention frame portion can be stretched to the linear shape shown in Example E for deployment through a deployment instrument. The retention frame portion may wind about or through the spherical space, or other spheroid-shaped space, in a variety of other manners.

The retention frame portion may be formed from a high modulus material or a low modulus material. Particularly, in embodiments in which the retention frame is formed from a relatively low modulus material, the retention frame may be formed into a configuration having a diameter and/or a shape that provides an appropriate spring constant. In one case, the retention frame portion may comprise a low modulus elastomer in a form having a spring constant without which the retention frame portion would otherwise experience significant deformation when subjected to the forces associated with urination. For example, the elastic wire of the retention frame may include one or more windings, coils, spirals, or combinations thereof, which may reduce the tendency of the elastic wire to deform during urination. In other words, the elastic wire may act as a spring due to the windings, coils, and/or spirals, even in cases in which the elastic wire is formed from a low modulus elastomer, such as polyurethane or silicone. The windings, coils, or spirals may be specifically designed to achieve a desirable spring constant. In various embodiments, the spring constant may be in the range of about 3 N/m to about 60 N/m. For example, the spring constant may be in the range of about 3 N/m to about 4 N/m, such as about 3.6 N/m to about 3.8 N/m. Such a spring constant may be achieved by one or more of the following techniques: increasing the diameter of the elastic wire used to form the frame, increasing the curvature of one or more windings of the elastic wire, and adding additional windings to the elastic wire. Example spring constants for certain low modulus wires are provided in Example 1, below.

The Material Portion

The material portion generally includes the material to be tested. The size, shape, and configuration of the material portion is selected so that the material can be associated with the retention frame portion, inserted into the body through a deployment instrument, and retained in the body for the duration of the study. Further, the size, shape, and configuration of the material portion may be selected based at least in part on the nature of the study and the characteristics of the material being investigated. In particular embodiments, the material portion includes a biomaterial, such as a urological material or other material to be tested in the bladder.

The material portion can be completely formed from a single material, or alternatively may be formed from a number of different materials. Forming the material portion from a number of discrete materials facilitates identifying how different materials respond upon exposure to the same implantation environment. The material portion also can include coatings, surface treatments, surface textures, or porosities, for testing purposes or other purposes.

The material portion also may include a drug, model drug, or the other agent releasable from the test material in vivo. The releasable agent may be disposed in, on, or throughout the test material, which may be a biopolymer material that is a candidate for construction of a medical device, for example.

In embodiments, the material portion is a tube-shaped structure having an interior bore or lumen for receiving the retention frame portion. The bore or lumen can be formed in a variety of manners, such as by punching, drilling, melting, extruding, or molding the material. The manner of forming the bore or lumen may be selected based at least in part on the mechanical and chemical properties of the material.

The size and shape of the material portion is selected at least in part to permit insertion through a deployment instrument. Particularly, an outer cross-sectional area of the material portion may not exceed an inner cross-sectional area of a deployment instrument designed for use with the animal model in which the device is implanted. The size and shape of the material testing portion, particularly about its exterior, also determines the surface area that is exposed to the implantation site for testing.

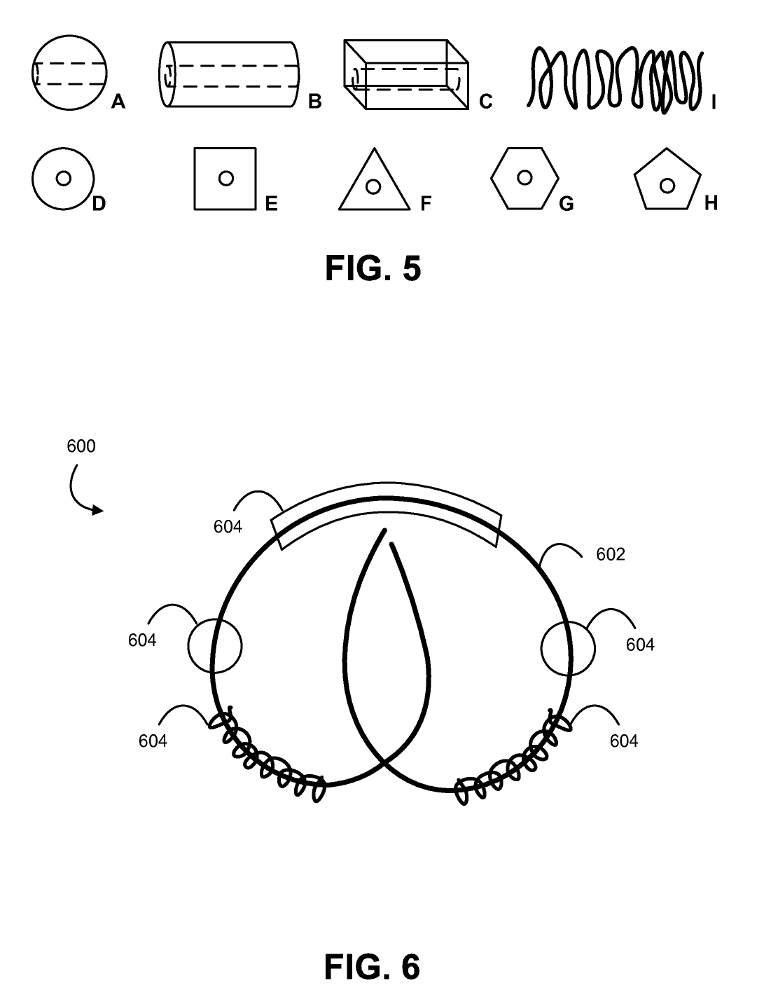

The material portion may have a wide variety of shapes, some of which are shown in FIG. 5 by way of example. Examples A, B, and C are perspective views of a spherical tube, a cylindrical tube, and a rectangular tube, respectively. The tubes may have other relative dimensions, including shorter, longer, wider, and narrower dimensions. Examples D through H illustrate cross-sectional shapes that can be used, including circular, square, triangular, pentagonal, and hexagonal shapes. Example I illustrates an embodiment of a material portion formed from a filament, string, or thread.

Combination of the Components

The retention frame portion is associated with the material portion to form the testing device. A variety of different associations are envisioned. For example, the material portion may surround or encase all or a part of the cross-section of the retention frame portion, or the material portion may be attached to one or more sides or ends of the retention frame portion, at either discrete points or along at least a portion of its length. The material portion also may be partially encased within the retention frame portion in some embodiments.

The material portion may be attached to the retention frame portion with any suitable biocompatible material or structure, such as with adhesive, a tie, a staple, a screw, or other mechanical fasteners, among others. The material portion also may be attached to the retention frame portion by surrounding or encasing at least a portion of the cross section of the retention frame portion, or the retention frame portion may at least partially extend through or be embedded within the material portion. The material portion also may wrap around the retention frame portion one or any number of times, or vice versa. Essentially, the material portion may be attached to any portion of the retention frame portion in any manner. Attachment also may not be required.

Additionally, a number of material portions may be associated with a single retention frame, depending on the configuration of the device. The different material portions may be formed from the same or different materials of construction, with the same or different sizes, shapes, porosities, surface textures, treatments, or coatings, among other alternatives and combinations thereof. Including multiple material portions may facilitate testing multiple different materials in the body, testing multiple different coatings on the same material, testing multiple different coating and material combinations, testing different characteristics of the same or different materials, such as sizes, textures or porosities, or a combination thereof.

Multiple discrete portions also may be attached to a single retention frame portion in cases in which the tested material does not have a suitable elasticity for moving with the retention frame portion between an insertion shape and a retention shape. In such case, the material may be connected to the retention frame in segments. The discrete testing portions can have the same or different shapes and can be spaced along the length of the retention frame portion at different locations.

An example is shown in FIG. 6, illustrating an embodiment of a device 600 having a retention frame portion 602 associated with multiple discrete material portions 604. The discrete material portions 604 have different configurations and are spaced along the length of the retention frame portion 604 at different locations, although other configurations are possible. In particular, the material portions 604 include a cylindrical tube that surrounds a portion of the retention frame portion 602, two spherical tubes positioned on opposite sides of the retention frame portion 602, and a two discrete filaments that wrap about the retention frame portion 602 and are attached using, for example, a medical grade adhesive, such as an implantable grade UV curable epoxy.

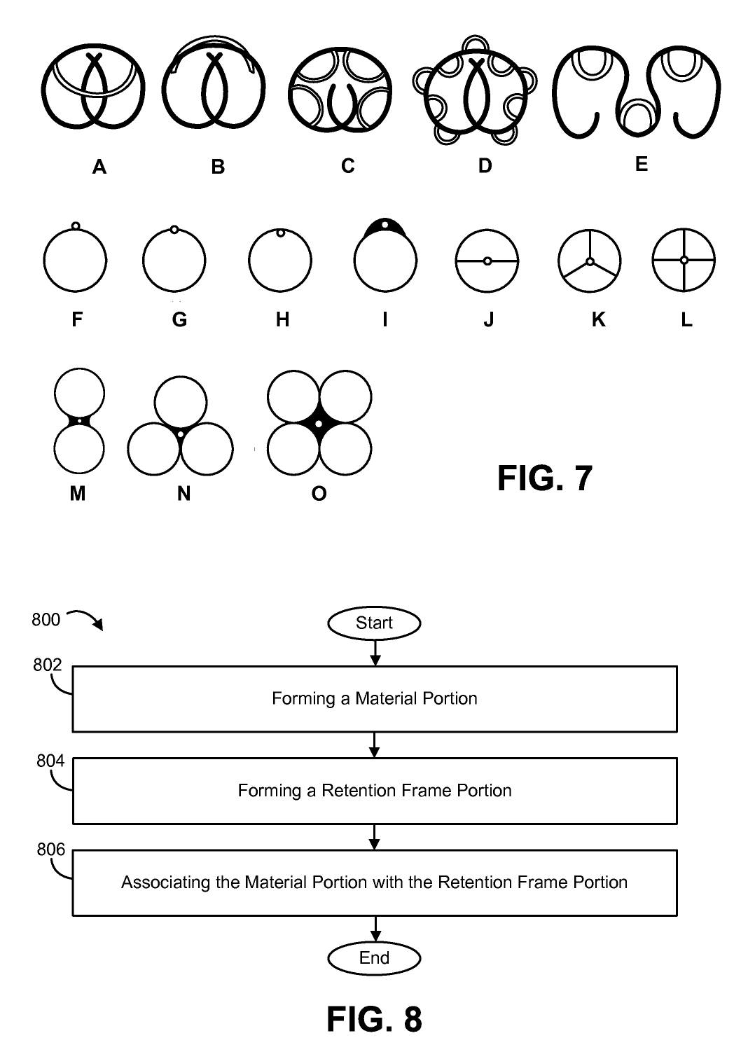

Additional example configurations are shown in FIG. 7 by way of example only, and combinations or obvious variations are included herein. The retention frame portion may have a pretzel shape as shown in Examples A through D, or other coiled, curved, or looped shapes, one example of which is shown in Example E. One material portion can be associated with the retention frame portion, as shown in Examples A and B, or multiple material portions can be associated with the retention frame portion, as shown in Examples C, D, and E. The material portion may have ends that attach to the retention frame portion, as shown in Examples A, C, D, and E, or the ends of the material portion may overlap the retention frame portion, as shown in Example B. The material portion may lie within the perimeter of the retention frame portion, as shown in Examples A, C, and E, outside of the perimeter of the retention frame portion, partially inside and partially outside the perimeter, as shown in Example B, or both inside and outside the perimeter, as shown in Example D.

The material portion and the retention frame portion also may be at least partially aligned, meaning the material portion may extend along at least a portion of the length of the retention frame portion, substantially parallel or coincident with the retention frame portion. For example, in FIG. 1, the material portion 102 extends along the entire length of the retention frame portion 104. The material portion 102 is a tubular material having an internal lumen or bore, and the retention frame portion 104 is an elastic wire inserted in the internal bore or lumen. In such case, the material portion has a suitable elasticity for following the shape of the retention frame portion when deformed for insertion through a catheter, cystoscope, or other deployment instrument.

FIG. 7 shows other examples wherein the material portion and retention frame portion at least partially align. Particularly, Examples F through L illustrate cross-sectional views of various embodiments having one or more material portions at least partially axially aligned with a single retention frame portion. The retention frame portion may extend along an exterior surface of the material portion as shown in Example F, an interior surface of the material portion as shown in Example G, or through the surface of the material portion as shown in Example H. The material portion may be strengthened near the retention frame portion with a reinforcement area, which may reduce the risk of the retention frame portion tearing through or becoming detached from the material portion, as shown in Example I. For example, the reinforcement area may be an area of a medical grade silicone adhesive. The retention frame portion also may be positioned within the interior of the urological material portion supported by a web, as shown in Examples J, K, and L. The web may permit associating the retention frame portion with multiple discrete material portions.

In still other embodiments, the retention frame portion may be associated with multiple material portions, extending along or between the material portions. Example M through O of FIG. 7 illustrate several alternative embodiments in cross-section. As shown, multiple discrete material portions may be joined together by a reinforcement area, with the retention frame portion embedded in the reinforcement area. Two, three, or four material portions may be used as shown in Examples M, N, and O, respectively, although additional material portions may be provided in embodiments not shown. The different material portions may be formed from the same or different materials of construction with the same or different surface textures, treatments or coatings, or any combinations of these and other alternatives.

The embodiments described above may be combined and varied to produce other testing devices that fall within the scope of the present disclosure.

Other Device Features

The device also may include end caps that blunt the ends of the device. The end caps also can prevent detachment of the material portions from the retention frame portion. Example end caps include beads formed on or covering ends of the device. The beads may be formed from adhesive, such from UV curable epoxy, or from a polymeric material that is attached to the device by friction fit or with adhesive. The end caps may be made at one end or both ends of a nitinol retention frame portion by plasma welding or laser welding. For example, an end cap can be formed on one end so that the material portion can be inserted over the retention frame portion before the other end cap is attached, either by friction fit or with adhesive.

In some embodiments, the device further includes at least one retrieval feature. The retrieval feature may facilitate removal of the device from the body. Examples include a string, a magnet, an O-shaped portion or a coiled portion, among others or combinations thereof. Embodiments of retrieval features are described in U.S. patent applications incorporated by reference above. In these and in other embodiments, the device may be retrieved using conventional endoscopic grasping instruments, such as alligator forceps, three or four-pronged optical graspers, or magnetized instruments, among others.

### II. Method of Making the Device

In another aspect, a method of making a material testing device is provided. FIG. 8 is a block diagram illustrating an embodiment of such a method 800. In block 802, a material portion is formed. In block 804, a retention frame portion is formed. In block 806, the material portion is associated with the retention frame portion.

In embodiments, the step of forming the material portion may include shaping the material. For example, an exterior surface of the material may be shaped to have the desired surface area exposed upon implantation. The test material may be made porous or non-porous. An interior lumen or bore also may be formed through the material portion. For example, the material portion may be formed by punching, drilling, melting, extruding, molding, or some combination thereof. Surface treatments or coatings also may be applied to the material portion for testing purposes or otherwise.

The step of forming a retention frame portion may vary depending on the material used to form the frame. In embodiments in which the retention frame comprises an elastic wire formed from a superelastic alloy or shape memory material, for example, the step of forming the retention frame may comprise forming the elastic wire into the relatively expanded shape and “programming” the shape into the elastic wire via heat treatment. For example, the retention frame portion 104 shown in FIG. 1 may be formed by forming the elastic wire into a pretzel shape and heat treating the elastic wire at a temperature over 500° C. for a period over five minutes. Also in such embodiments, forming the retention frame portion may include one or more of the following: forming a polymer coating or sheath over at least a portion of the elastic wire, smoothening the ends of the elastic wire, and applying a radio-opaque material to at least a portion of the elastic wire. In such embodiments the polymer sheath, the radio-opaque material, and the smoothening material may be applied to the elastic wire in any order. For example, the polymer sheath or coating may be placed over the elastic wire, a platinum wire may be wound around ends of the elastic wire to improve the radio-opacity of the device to x-ray, and the ends of the elastic wire may be smoothened with an ultraviolet-curable epoxy. However, one or more of these steps may be omitted. For example, the polymer sheath may be omitted in embodiments in which the testing portion covers or coats the retention frame portion.

In embodiments, the step of forming the retention frame may comprise forming one or more windings, coils, loops or spirals in the frame so that the frame functions as a spring. For example, in embodiments in which the retention frame comprises a low modulus elastomer, the retention frame may be formed by extrusion, liquid injection molding, transfer molding, or insert molding, among others.

The step of associating the material portion with a retention frame portion may comprise orienting the material portion with reference to the retention frame portion and applying an adhesive or a mechanical fastener there between. The material portion may be oriented in a variety of orientations as described above. In other embodiments, the step of associating the material portion with the retention frame portion may comprise inserting an elastic wire of the retention frame portion at least partially through the material portion. In still other embodiments, the step of associating the material portion with the retention frame portion may comprise integrally forming the two portions together.

### III. Use and Applications of the Device

The material testing device may be used to test materials locally in essentially any site of an animal. In one embodiment, a method of testing a material in an animal includes: associating the material with a retention frame to form a testing device, the retention frame movable between a first shape suited for insertion through a deployment instrument and a second shape suited for retention in the animal; inserting the testing device in the first shape into the deployment instrument; driving the testing device through the deployment instrument into the animal; permitting the testing device to assume the second shape once in the animal so that the testing device is retained in the animal for a testing period; removing the testing device from the animal after the testing period is complete; and analyzing the material and/or biological tissue at or about the deployment site of the device in the animal.

In a preferred embodiment, the material may be implanted in a mammal that is suited for testing materials intended for use in humans, such as a rabbit, a cat, a pig, a dog, or a sheep, among others, including humans. In a preferred embodiment, the material is implanted/deployed in the bladder of the animal. In other embodiments, the material may be implanted in another genitourinary site, body cavity, or lumen of the animal. For example, the device may be implanted in a space in the vagina, a gastric cavity, the peritoneal cavity, or an ocular cavity. The device is retained in the implantation site over a testing period (e.g., two, three, or four weeks, a month, or more), which may be predetermined. For example, the testing period may be between about 1 day and about 90 days. The device is then retrieved from the animal so that the material can be analyzed. The device can be retrieved using a deployment inserted through the urethra, for example. In some cases, necropsy is performed. In this and in other cases, tissue about the implantation site may be collected for additional analysis. For example, bladder tissue may be collected. The material may be analyzed for biofilm formation, encrustation, degradation, and/or bacterial adhesion and colonization, among others or combinations thereof. The laboratory animal also may be analyzed for reaction to the material, such as infection or necrosis, among others.

The device may be implanted in the implantation site, such as the bladder, using any suitable deployment device. Examples include a catheter, a urethral catheter, or a cystoscope, whether commercially available or specially developed for this purpose.

In one example, a method of implanting a material testing device in a body comprises passing the material testing device through a deployment instrument in a deployment shape and releasing the device from the deployment instrument into the body, the device assuming a relatively expanded shape once the device emerges from the deployment instrument for retention in the body. In embodiments, the deployment shape is a relatively lower profile shape, which may be a relatively linear, folded, expanded, or compressed form. The retention shape is an expanded shape having one of the configurations described above, or otherwise.

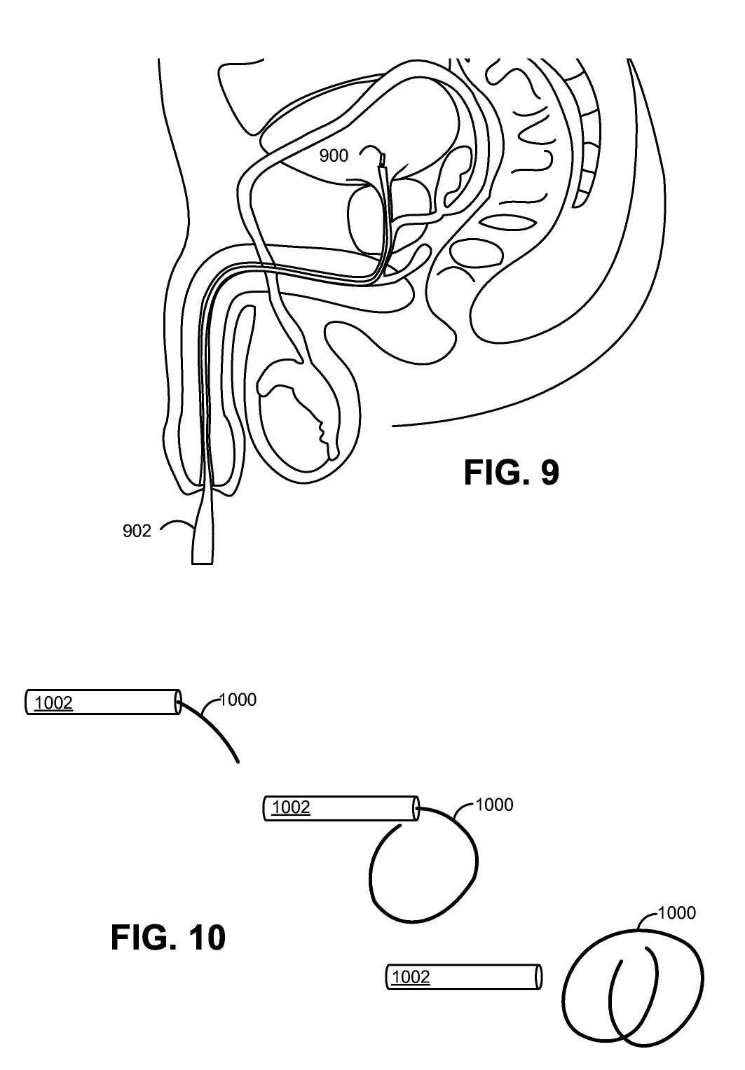

In particular embodiments, the method implants a urological material testing device in the bladder. The deployment instrument may be inserted into the urethra to permit access to the bladder. An example is shown in FIG. 9, which illustrates a deployment instrument 902 navigating the urethra to the bladder. The anatomy of a male human is shown by way of example only. A testing device 900 is shown exiting the deployment instrument into the bladder, for the purpose of testing a urological material therein.

FIG. 10 illustrates an embodiment of the releasing step of the method. As shown in FIG. 10, the device 1000 may change shape as it emerges from the deployment instrument 1002, returning to the relatively expanded shape for retention in the body, such as in the bladder.

In one embodiment, the device 1000 may be driven through the deployment instrument using a stylet. In another embodiment, the device may be driven by a flow of fluid, such as a water-based lubricant, into the implantation site. In such embodiments, the device is loaded into a deployment instrument, preferably from the distal side. The fluid is then forcefully injected from the proximal side of the deployment instrument, such as using a syringe, so that the fluid pushes the device through the deployment instrument until it is ejected into the implantation site. Methods of implantation, and devices specially designed for implantation, are further described in U.S. patent applications incorporated by reference above.

### Example 1

### Sample Spring Constants for Certain Low Modulus Wires

A nitinol wire having a Young's modulus of about 30 GPa, a diameter of about 0.2286 mm, an arc radius of about 1.5 cm, and one coil may have a spring constant of about 3.7 N/m. A polyurethane wire having a Young's modulus of about 25 MPa, a diameter of about 1 mm, an arc radius of about 1 cm, and one coil may have a spring constant of about 3.8 N/m. A silicone wire having a Young's modulus of about 2.41 MPa, a diameter of about 1.2 mm, an arc radius of about 0.75 cm, and two coils may have a spring constant of about 3.6 N/m. A poly(glycerol-sebacate) (PGS) wire having a Young's modulus of about 1.7 MPa, a diameter of about 1.2 mm, an arc radius of about 0.76 cm, and three coils may have a spring constant of about 3.7 N/m.

Publications cited herein and the materials for which they are cited are specifically incorporated by reference. Modifications and variations of the methods and devices described herein will be obvious to those skilled in the art from the foregoing detailed description. Such modifications and variations are intended to come within the scope of the appended claims.

What is claimed is:

1. A method of testing a material in an animal, the method comprising:
associating the material with a retention frame to form a testing device, the retention frame movable between a first shape suited for insertion through a deployment instrument and a second shape suited for retention in the animal;
inserting the testing device in the first shape into the deployment instrument;
driving the testing device through the deployment instrument into the animal;
permitting the testing device to assume the second shape once in the animal so that the testing device is retained in the animal for a testing period;
removing the testing device from the animal after the testing period is complete; and
analyzing the material.

2. The method of claim 1, wherein analyzing the material comprising analyzing the material for one or more of the following: biofilm formation, encrustation, or degradation.

3. The method of claim 1, further comprising analyzing the animal for infection or other reactions to the material.

4. The method of claim 1, wherein the testing period is between about 1 day and about 90 days.

5. The method of claim 1, further comprising collecting tissue from the animal and analyzing the tissue.

6. A method for minimally invasive in vivo testing of a material comprising:
deploying a testing device into the bladder of a mammalian laboratory animal via a deployment instrument inserted through the urethra, wherein the testing device comprises one or more test materials; and
leaving the device in the bladder for a period effective to achieve a study objective concerning the one or more test materials; and
removing the testing device from the bladder.

7. The method of claim 6, further comprising analyzing the one or more test material for biofilm formation, encrustation, or degradation.

8. The method of claim 6, further comprising analyzing the mammalian laboratory animal for infection or other reactions to the one or more test materials.

9. The method of claim 6, wherein the period is from 1 day to 90 days.

10. The method of claim 6, wherein the device is removed from the bladder via a deployment instrument inserted through the urethra or via necropsy.

11. The method of claim 6, further comprising collecting tissue from the bladder and analyzing the tissue.

12. A material testing device for testing a material in a body cavity of an animal, comprising:
a retention frame portion suited for retaining the device in the body cavity; and
at least one material portion associated with the retention frame portion for testing in the body cavity.

13. The material testing device of claim 12, wherein the at least one material portion comprises a first material portion and a second material portion, the first material portion having at least one of the following differences from the second material portion: material, size, shape, surface texture, surface treatment, or porosity.

14. The material testing device of claim 13, wherein the first and second material portions are discrete units.

15. The material testing device of claim 12, wherein the retention frame portion comprises an elastic wire.

16. The material testing device of claim 12, wherein the retention frame portion comprises a superelastic alloy or shape memory material.

17. The material testing device of claim 12, wherein the retention frame portion comprises a nitinol wire.

18. The material testing device of claim 12, wherein the retention frame portion has a spring constant in the range of about 3 N/m to about 4 N/m.

19. The material testing device of claim 12, wherein the retention frame portion comprises one or more windings, coils, spirals, loops, curls or sub-circles.

20. The material testing device of claim 12, wherein the at least one material portion comprises a polymeric material.
